(19) United States
(12) Patent Application Publication
(10) Pub. No.: US 2011/0230771 A1
(43) Pub. Date: Sep. 22, 2011

(54) HEART FAILURE STATUS MONITORING

(75) Inventors: Steve Koh, South Pasadena, CA (US); Euljoon Park, Valencia, CA (US)

(73) Assignee: PACESETTER, INC., Sylmar, CA (US)

(21) Appl. No.: 12/728,064

(22) Filed: Mar. 19, 2010

(51) Int. Cl. A61B 5/02; A61B 5/01

(52) U.S. Cl. 600/485; 600/508; 600/549

(57) ABSTRACT

Left atrial pressure and temperature of a patient are monitored to identify a normal wake state, a normal sleep state, and any deviation from those normal states (e.g., an alarm state). In the event an alarm state is identified, a determination is made as to whether to generate an indication of heart failure exacerbation based on a heart failure score. In addition, congestion and perfusion in a patient may be monitored over time to provide a two-dimensional indication of a trend relating to the heart failure status of the patient.

### TECHNICAL FIELD

This application relates generally to implantable medical devices and more specifically, but not exclusively, to techniques for monitoring heart failure status.

### BACKGROUND

Heart failure is a debilitating disease in which abnormal function of a patient's heart leads to inadequate blood flow to the patient's body. While a heart failure patient may not suffer debilitating symptoms immediately, with few exceptions, the disease is relentlessly progressive. Moreover, as heart failure progresses, it may become increasingly difficult to manage. Accordingly, a need exists for effective techniques for monitoring heart failure status so that appropriate treatment may be prescribed for the patient in a timely manner.

### SUMMARY

A summary of several sample aspects of the disclosure follows. It should be appreciated that this summary is provided for the convenience and does not wholly define the breadth of the disclosure. For convenience, the term “some aspects” may be used herein to refer to a single aspect or multiple aspects of the disclosure.

The disclosure relates in some aspects to monitoring left atrial pressure (LAP) and temperature of a patient to identify a normal wake state, a normal sleep state, and any deviation from those normal states (e.g., a transition to an alarm state). In the event an alarm state is identified, a determination is made as to whether to generate an indication of heart failure exacerbation based on an alarm state score. For example, if the monitoring results in several recent identifications of the alarm state (e.g., indicative of acute heart failure), a message may be sent to trigger a change in therapy to treat heart failure exacerbation. In addition, if the monitoring results in a larger number of identifications of the alarm state (e.g., indicative of chronic heart failure), a message may be sent to trigger urgent care for the patient.

The disclosure relates in some aspects to repeatedly monitoring congestion and perfusion in a patient over time to provide a two-dimensional (2-D) indication of a trend relating to the heart failure status of the patient. For example, a two-dimensional graph may be displayed to indicate changes in the patient's congestion and perfusion conditions over time. In this way, more appropriate therapy may be provided for the patient since the effects of the current therapy or changes in therapy may be more readily determined.

### BRIEF DESCRIPTION OF THE DRAWINGS

These and other features, aspects, and advantages will be more fully understood when considered with respect to the following detailed description, the appended claims, and the accompanying drawings, wherein:

FIG. 1 is a simplified block diagram illustrating sample components for monitoring heart failure condition;

FIG. 2 is a simplified diagram illustrating sample sleep, wake, and alarm states;

FIG. 3 is a simplified flowchart of sample operations that may be performed to generate an indication of heart failure status based on collected LAP and temperature information;

FIGS. 4A-4D are simplified diagrams illustrating sample temperature information, sample LAP information, and corresponding sleep and wake states;

FIGS. 5A-5D are simplified diagrams illustrating sample temperature information, sample LAP information, and a corresponding alarm state;

FIGS. 6A-6D are simplified diagrams illustrating sample temperature information, sample LAP information, and a corresponding critical alarm state;

FIG. 7 is a simplified flowchart of sample operations that may be performed to generate different indications of heart failure status based on collected LAP and temperature information;

FIG. 8 is a simplified state diagram illustrating sample score modification during sleep, wake, and alarm states;

FIG. 9 is a simplified state diagram illustrating sample sleep, wake, and alarm states, including temperature-based score modification;

FIG. 10 is a simplified flowchart of sample operations that may be performed to provide a two-dimensional indication of heart failure status based on collected congestion and perfusion information;

FIG. 11 is a simplified two-dimensional diagram illustrating a sample heart failure status trend based on collected congestion and perfusion information;

FIG. 12 is a simplified flowchart of sample operations that may be performed to provide indications of heart failure status based on collected congestion and perfusion information;

FIG. 13 is a simplified diagram of an embodiment of a monitoring system including an implantable medical device and an external monitor device;

FIG. 14 is a simplified diagram of an embodiment of an implantable stimulation device in electrical communication with one or more leads implanted in a patient's heart for sensing conditions in the patient, delivering therapy to the patient, or providing some combination thereof; and

FIG. 15 is a simplified functional block diagram of an embodiment of an implantable cardiac device, illustrating basic elements that may be configured to sense conditions in the patient, deliver therapy to the patient, or provide some combination thereof.

In accordance with common practice the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

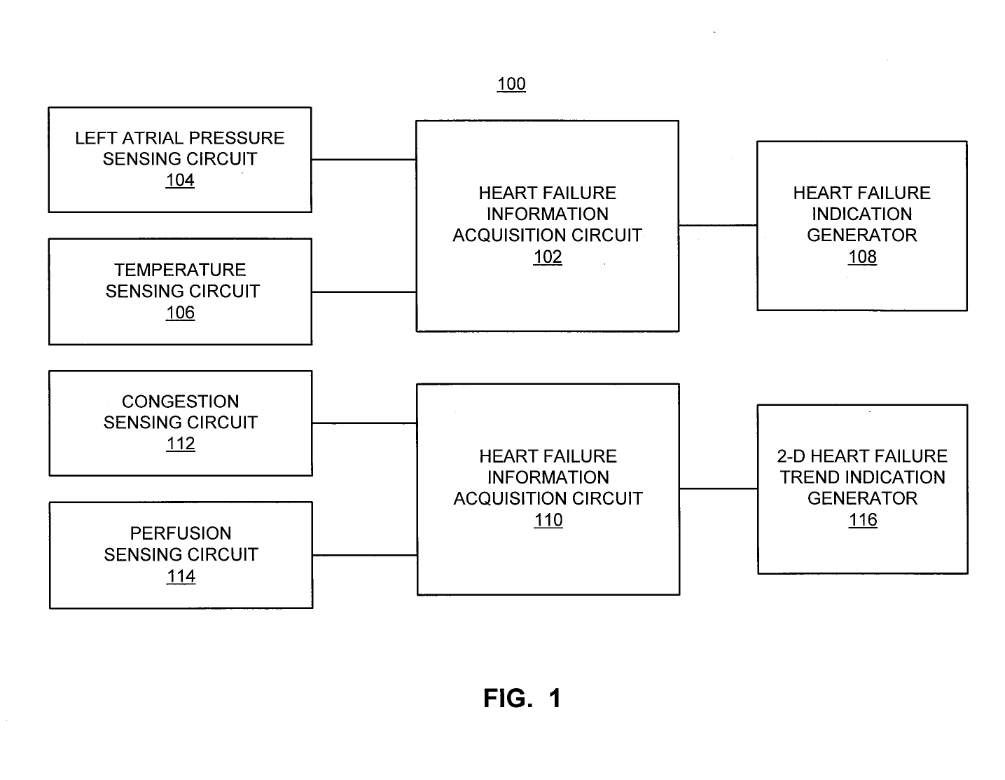

FIG. 1 illustrates sample functional components that may be employed to provide heart failure monitoring in accordance with the teachings herein. Two different monitoring systems are depicted in FIG. 1. A monitoring system corresponding to blocks 102-108 provides a heart failure indication based on LAP and temperature information. A monitoring system corresponding to blocks 110-116 provides a heart failure indication based on congestion and perfusion information. These monitoring systems may be deployed in different devices in some implementations and in the same device in other implementations.

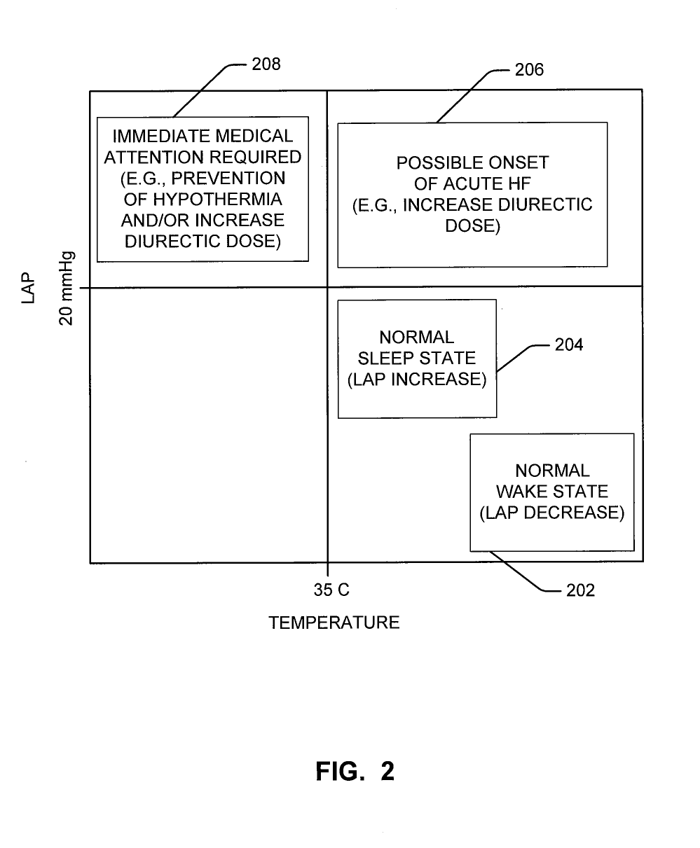

A heart failure information acquisition circuit 102 collects LAP information from an LAP sensing circuit 104 and temperature information from a temperature sensing circuit 106 and provides the collected information to a heart failure indication generator 108. The heart failure indication generator 108 generates an indication of heart failure status (e.g., acute heart failure or chronic heart failure) based on the collected information. This aspect of the disclosure is described in more detail below in conjunction with FIGS. 2-9.

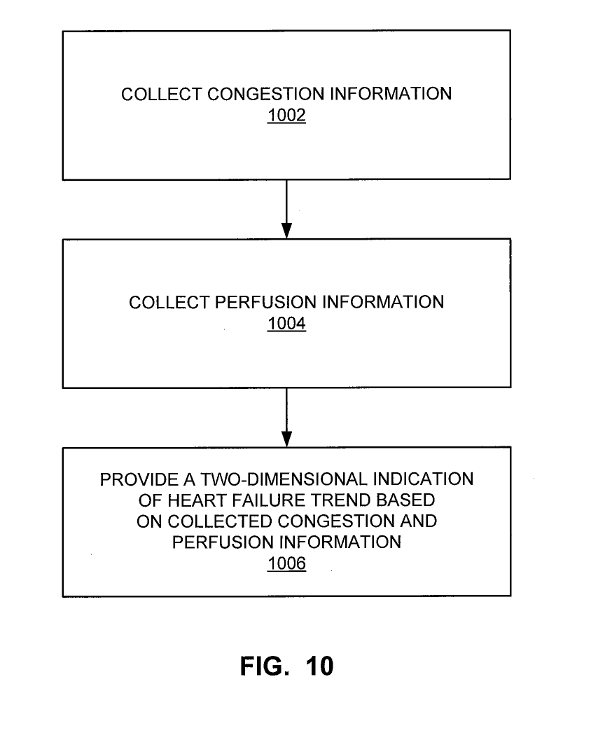

A heart failure information acquisition circuit 110 collects congestion information from a congestion sensing circuit 112 and perfusion information from a perfusion sensing circuit 114 and provides the collected information to a 2-D heart failure trend indication generator 116. The 2-D heart failure trend indication generator 116 generates a 2-D indication of a heart failure status trend (e.g., a 2-D plot covering a period of several weeks or months) based on the collected information. This aspect of the disclosure is described in more detail below in conjunction with FIGS. 10-12.

Referring initially to FIGS. 2-9, the disclosure relates in some aspects to detecting heart failure exacerbation and ensuing hypothermia using circadian rhythm derived from temperature (e.g., cardiac core temperature) and LAP measurements. This process relies, in part, on the observation that body core temperature reflects metabolic demand (oxygen consumption) and thus may be used for circadian detection. Specifically, a record is maintained as to how much a patient's body temperature drops when the patient is asleep and increases when the patient is awake. In addition, a record is maintained as to how much LAP drops when a patient is in a sitting or standing posture and increases when the patient is in a supine posture (e.g., during sleep). Thus, by monitoring a patient's temperature and LAP and identifying when these parameters fall outside of normal wake and sleep values, it is possible to detect heart failure progression or detect dangerous end-stage heart failure exacerbation accompanied by hypothermia.

FIG. 2 illustrates sample temperature and LAP borderlines that may be employed to monitor a patient's heart failure status. Here, a normal wake state may be associated with a data set including temperature and LAP values in an area represented by block 202. In addition, a normal sleep state may be associated with a data set including temperature and LAP values in an area represented by block 204. Here, it may be seen that the temperature values associated with the normal sleep state are slightly lower than those associated with the normal wake state. In addition, the LAP values associated with the normal sleep state are slightly higher than those associated with the normal wake state.

FIG. 2 also illustrates alarm states that may be defined when the patient's LAP increases. For example, an alarm state associated with increased LAP (e.g., LAP greater than 20 mmHg) may be associated with a data set including temperature and LAP values in an area represented by block 206. In some aspects, this alarm state may reflect acute heart failure. For example, the possible onset of acute heart failure may be indicated when a patient has temperature and LAP readings that correspond to the values represented by block 206. In such a case, an indication of acute heart failure may be generated that indicates, for example, that the patient's therapy should be modified. For example, a message may be sent to the patient, a medical provider, or some other entity indicating that a diuretic dose for the patient should be increased.

In addition, an alarm state associated with increased LAP (e.g., LAP greater than 20 mmHg) and lower temperature (e.g., less than 35 C) may be associated with a data set including temperature and LAP values in an area represented by block 208. In some aspects, this alarm state may reflect chronic heart failure. For example, the need for immediate medical attention may be indicated when a patient has temperature and LAP readings that correspond to the values represented by block 208. In such a case, it may be desirable to generate an urgent indication of chronic heart failure to signal that the patient should receive immediate medical attention. For example, a message may be sent to the patient, a medical provider, or some other entity indicating that immediate treatment for hypothermia is required and/or that the diuretic dosage prescribed for the patient should be increased immediately.

It should be appreciated that FIG. 2 illustrates sample borderlines and that other borderlines may be applied in other cases. In some cases these borderlines may be patient specific. For example, data sets 202-208 for a given patient may be defined based on empirical measurements made while the patient is awake, sleeping, and possibly while experiencing episodes of heart failure. In addition, a treating physician may define the temperature and LAP values that correspond to the acute and chronic alarm states for that patient. Also, in some implementations a different number of states may be defined (e.g., one alarm state or more than two alarm states).

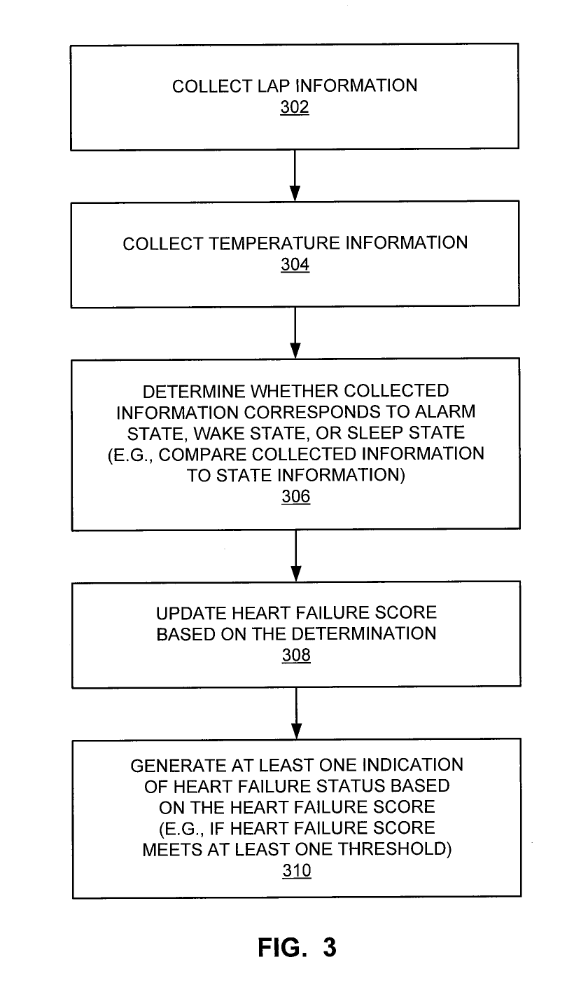

FIG. 3 illustrates sample operations that may be performed to provide an indication of heart failure status based on LAP information and temperature information collected from the patient. For convenience, the operations of FIG. 3 (or any other operations discussed or taught herein) may be described as being performed by specific components (e.g., components of FIG. 1, 13, or 15). It should be appreciated, however, that these operations may be performed by other types of components and may be performed using a different number of components. It also should be appreciated that one or more of the operations described herein may not be employed in a given implementation.

As represented by blocks 302 and 304, LAP information and temperature information are collected. For example, an implantable medical device may employ sensing circuits and/or may be coupled to other sensing circuits (e.g., implantable sensing circuits) that sense signals from which LAP information and temperature information may be derived.

As represented by block 306, a determination is made as to whether the collected information corresponds to an alarm state, a wake state, or a sleep state. For example, the information collected at blocks 302 and 304 may be compared to data sets that are maintained for the alarm, wake, and sleep states. As mentioned above, these data sets may be generated empirically by collecting temperature and LAP information from the patient at times that are known or expected to correspond to the alarm, wake, and sleep states.

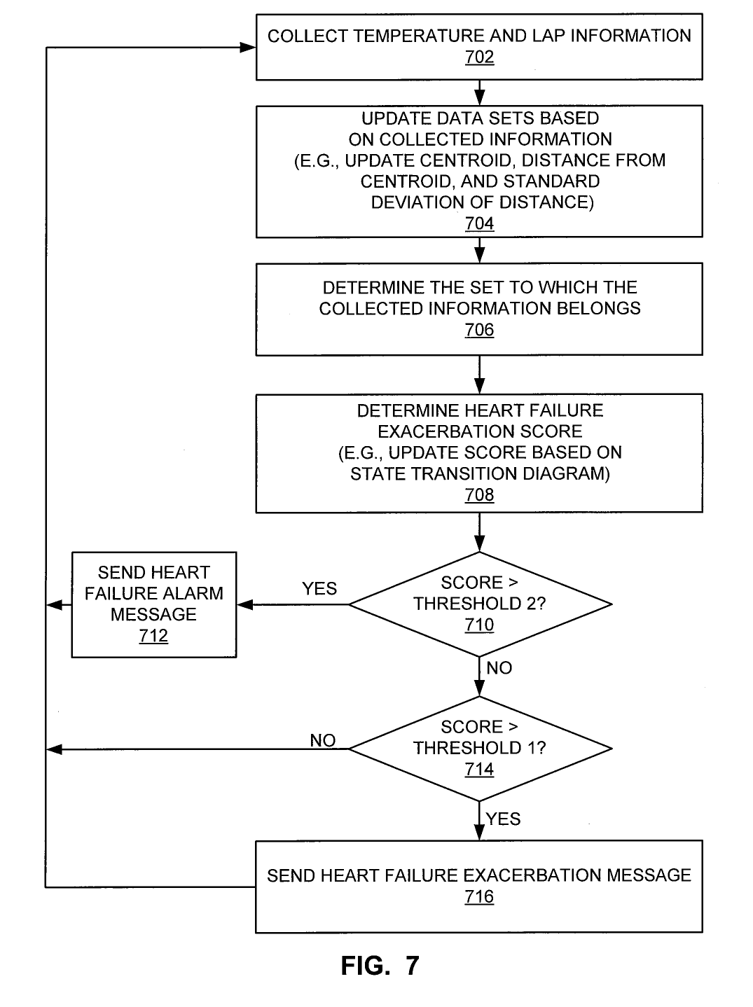

As represented by block 308, a heart failure score is updated based on the determination of block 306. For example, as discussed in more detail below in conjunction with FIGS. 7-9, a heart failure score may be increased whenever the alarm state is detected (e.g., when the determination at block 306 corresponds to an alarm state). Conversely, the heart failure score may be decreased whenever there is a transition from an alarm state to a normal state (e.g., sleep or wake state). Accordingly, the heart failure score may provide an indication as to whether a patient is experiencing heart failure exacerbation.

Thus, as represented by block 310, one or more indications of heart failure status may be generated based on the updated heart figure score. For example, an indication may be generated (e.g., a message sent) if the heart failure score meets a threshold (e.g., equals or exceeds a threshold). In addition, as discussed in more detail below in conjunction with FIG. 7, in some implementations more than one threshold may be employed whereby different indications may be generated when the heart failure score meets these different thresholds.

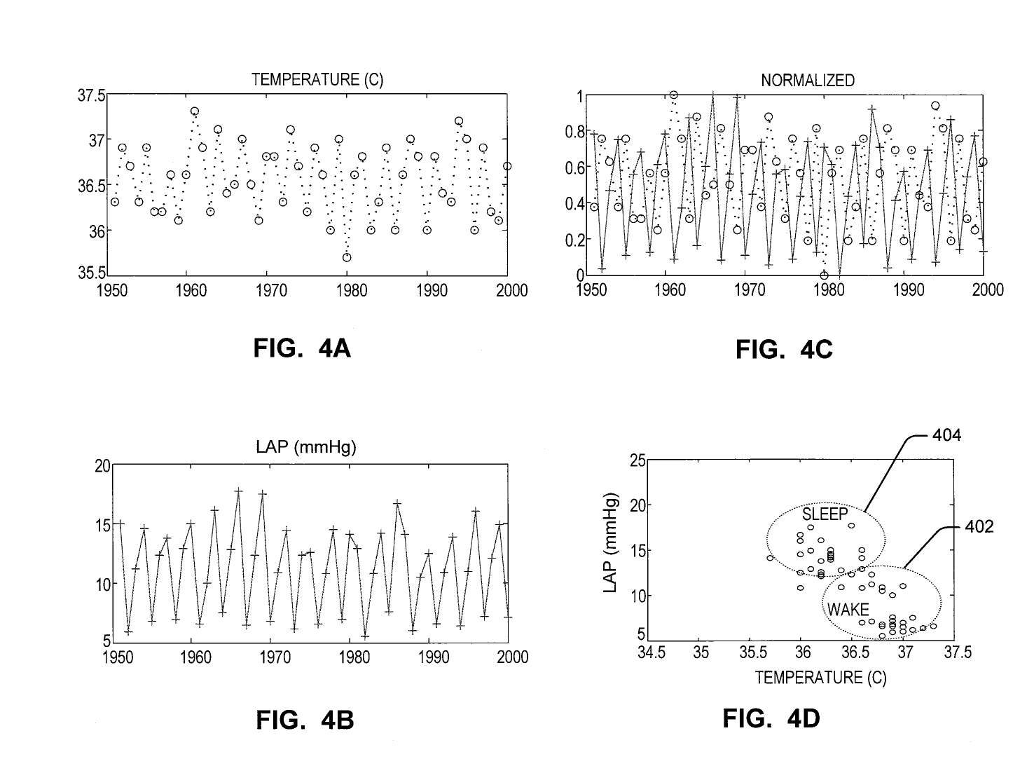

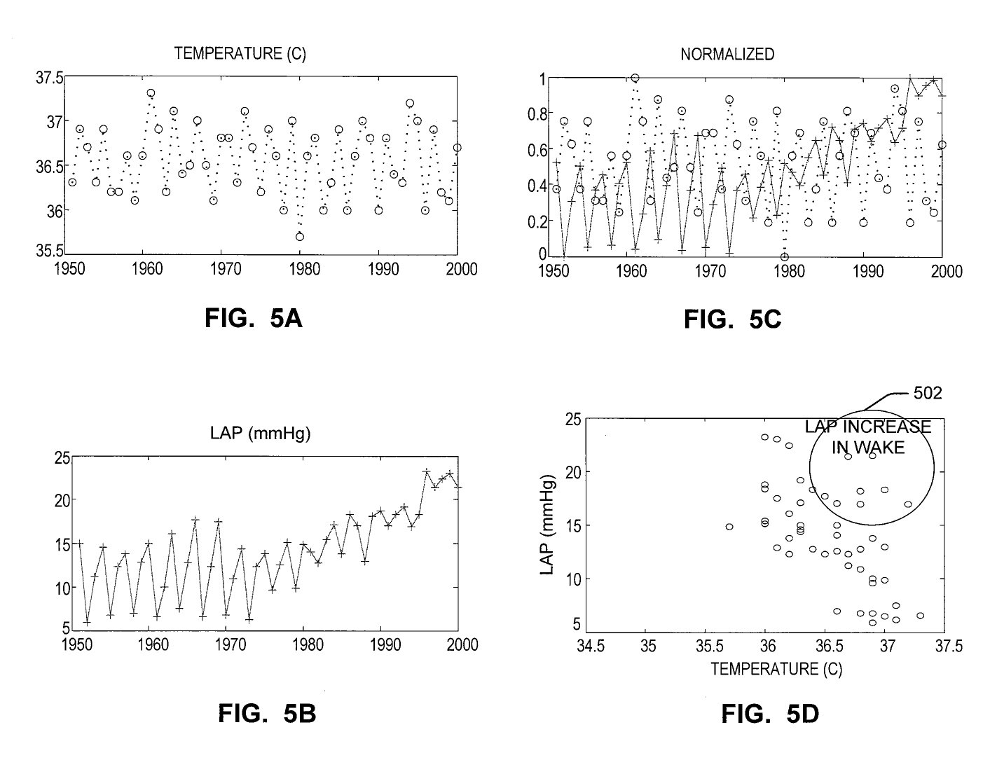

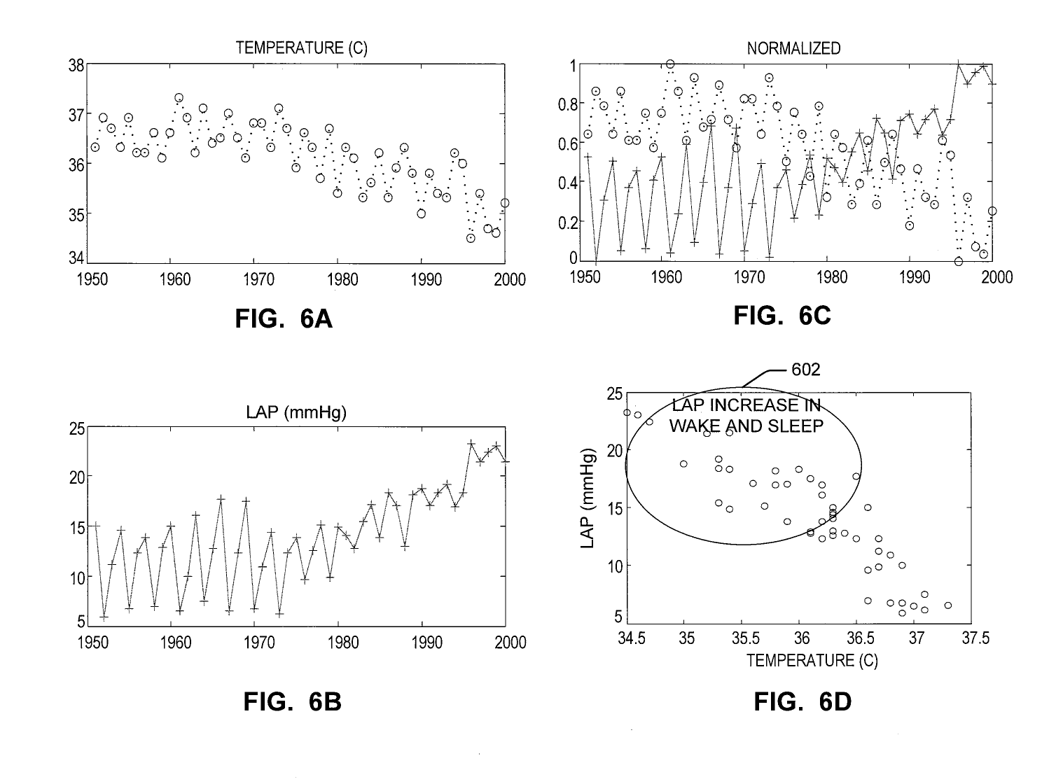

FIGS. 4A-6D illustrates how sample data sets corresponding to alarm, wake and sleep states may be defined. Specifically, FIGS. 4A-4D illustrate sample data sets for normal sleep and wake states. FIGS. 5A-5D illustrate a sample data set for an acute alarm state. FIGS. 6A-6D illustrate a sample data set for a chronic alarm state.

Referring initially to FIGS. 4A and 4B, these figures illustrate an example of temperature and LAP values that may be collected over a period of time when a patient is not experiencing heart failure exacerbation. For example, FIG. 4A illustrates temperature samples (indicated by the circles) for a period of several days. FIG. 4B illustrates LAP samples (indicated by the crosses) taken over the same time period. These figures illustrate that daily circadian LAP and temperature are oscillatory in this normal state (without heart failure exacerbation). For example, temperatures above approximately 36.5 C may correspond to a wake state while temperatures below approximately 36.5 C may correspond to a sleep state. In addition, LAP above approximately 12 mmHg may correspond to a sleep state while LAP below approximately 12 mmHg may correspond to a wake state.

FIG. 4C provides a normalized representation of the charts of FIGS. 4A and 4B. This figure clearly illustrates the out-of-phase nature between temperature and LAP.

FIG. 4D is a 2-D plot of the samples from FIGS. 4A and 4B. Here, it may be seen that due to the out-of-phase nature of temperature and LAP, the values associated with the sleep state and the wake state are located in two substantially distinct regions in the 2-D plane. Here, temperatures above approximately 36.5 C and LAP below approximately 12 mmHg generally correspond to the wake state as represented by the data values approximately within area 402. In addition, temperatures below approximately 36.5 C and LAP above approximately 12 mmHg correspond to the sleep state as represented by the data values approximately within area 404.

Referring now to FIGS. 5A-5D, at the onset of heart failure exacerbation, body core temperature may tend to oscillate according to the circadian rhythm without significant mean change. However, LAP may slowly increase, whereby the peak-to-valley LAP amplitude decreases with the increase in mean LAP.

FIGS. 5A and 5B illustrate estimated temperature samples and LAP samples for a period of several days when a patient is experiencing the onset of acute heart failure. Here, the temperature samples of FIG. 5A correspond to the temperature samples of FIG. 4A. FIG. 5B illustrates, however, that mean LAP may increase over time at the onset of acute heart failure exacerbation (e.g., at samples 1975-2000). FIG. 5C provides a normalized representation of the charts of FIGS. 5A and 5B.

FIG. 5D is a 2-D plot of the samples from FIGS. 5A and 5B. Here, as represented by the data values approximately within area 502, it may be seen that there is an increase in LAP (particularly when the patient is awake).

Referring now to FIGS. 6A-6D, it is known that body temperature decreases in the case of severe chronic heart failure. The reasons for this decrease in body temperature include: 1) poor circulation which causes an imbalance between heart production and dissipation; and 2) accumulation of carbon dioxide in the blood. In the latter case, an increase in carbon dioxide will increase the pH of the blood as the blood buffers the carbon dioxide, thereby generating [H+] ions. This increased acidity, in turn, will reduce the efficiency at which the body uses oxygen. In general, heart failure in combination with hypothermia requires immediate medical attention. For example, there may be a sudden temperature decrease when LAP increases near 30 mmHg. If this psychological condition persists, the patient may die unless immediate intervention is performed

FIGS. 6A and 6B illustrate estimated temperature samples and LAP samples for a period of several days when a patient is experiencing chronic heart failure. Here, it may be seen that there is a decrease in temperature and increase in LAP (e.g., at samples 1975-2000). FIG. 6C provides a normalized representation of the charts of FIGS. 6A and 6B.

FIG. 6D is a 2-D plot of the samples from FIGS. 6A and 6B. Here, as represented by the data values approximately within area 602, it may be seen that there is a decrease in temperature and an increase in LAP both when the patient is awake and when the patient is asleep.

In view of the above, a heart failure monitoring scheme according to the teachings herein may use body core temperature as a surrogate for determining circadian wake or sleep states, wherein the interpretation of LAP information is different based on the circadian state associated with the collection of the LAP information. Here, upon collection of the LAP and temperature information, a 2-D Cartesian decision grid (temperature and LAP) may be defined. This decision grid may be subdivided into four regions (e.g., wake state, sleep state, alarm state, and don't care). A scoring method based on state transitions (e.g., between wake, sleep, and alarm states) may then be employed to determine whether the identification of alarm state corresponds to acute heart failure or chronic heart failure. For example, tiered thresholds may be employed whereby a first threshold corresponds to acute heart failure and a second threshold corresponds to chronic heart failure. In some implementations, if the first threshold is met, a message is sent (e.g., to the patient) to request an increase in diuretic dosage. Conversely, if the second threshold is met, an urgent message may be sent (e.g., to a monitoring station) to indicate heart failure-induced hypothermia. In the latter case, the message may indicate that immediate treatment is required.

FIG. 7 illustrates a sample monitoring process employing tiered thresholds in accordance with the teachings herein. This process may be performed on a repeated basis (e.g., hourly, twice a day, once a day, or at other times).

As represented by block 702, temperature and LAP information is collected at some point in time. For example, an implantable medical device may be configured to collect temperature and LAP information on a repeated basis (e.g., continuously, hourly, twice a day, or at other times) that may or may not coincide with the timing of the operations of blocks 708-716.

The temperature information may be collected in various ways. For example, an implantable medical device may include a temperature sensing circuit and/or may be coupled to a temperature sensing circuit. As an example of the latter case, a temperature sensing circuit may be implanted in a patient and coupled to the implantable medical device via an implantable lead or via a wireless communication link.

A temperature sensing circuit may take various forms. For example, a temperature sensing circuit may comprise a temperature sensor. In addition, in some implementations a temperature sensing circuit may sense signals from which temperature information may be determined (e.g., estimated). In addition, other types of information may be used instead of temperature in some cases (e.g., in implementations that do not use a temperature sensor). For example, activity information (e.g., as provided by an activity sensor) may be used instead of temperature information in some implementations. Here, the level of patient activity may indicate when a patient is awake or asleep, and a prolonged lack of activity may indicate chronic heart failure since a patient with chronic heart failure may become very inactive.

The LAP information also may be collected in various ways. For example, an implantable medical device may include a LAP sensing circuit and/or may be coupled to a LAP sensing circuit. As an example of the latter case, a LAP sensing circuit may be implanted in a patient and coupled to the implantable medical device via an implantable lead or via a wireless communication link.

A LAP sensing circuit may take various forms. In some implementations a LAP sensing circuit comprises a pressure sensor (e.g., implanted in or near the left atrium). For example, an implanted lead with a pressure sensor on its distal end may be routed to the right atrium and through the atrial septum into the left atrium. In some implementations a temperature sensing circuit may sense signals from which LAP information may be determined (e.g., estimated). For example, LAP may be estimated based on right ventricle pressure or other sensed signals.

As represented by block 704, one of data sets corresponding to the alarm, sleep, and wake states is updated based on the new information collected at block 702. For example, the newly collected temperature and LAP values may be added to a data set having similar values.

Other information may be taken into account to update the data sets in some implementations. For example, it may be determined (e.g., estimated) whether the patient is awake or asleep when a new set of data was collected based on activity, posture, time of day, or other information. Accordingly, this information may be used to identify the proper alarm, wake, or sleep data set to be updated with the new set of data.

A data set may take various forms. For example, in some implementations a data set may be defined in terms of one or more of a centroid, distance from the centroid, or standard deviation of the distance. Accordingly, as a new set of data (e.g., the current temperature and LAP values) is added to a data set, one or more of the centroid of the data set, distance from the centroid, or standard deviation of distance may be updated,

As represented by block 706, a determination is made as to which data set the newly collected information belongs. For example, the newly collected temperature and LAP values may be compared to the alarm, wake, and sleep data sets to determine whether the newly collected values correspond to an alarm state, a normal wake state, or a normal sleep state.

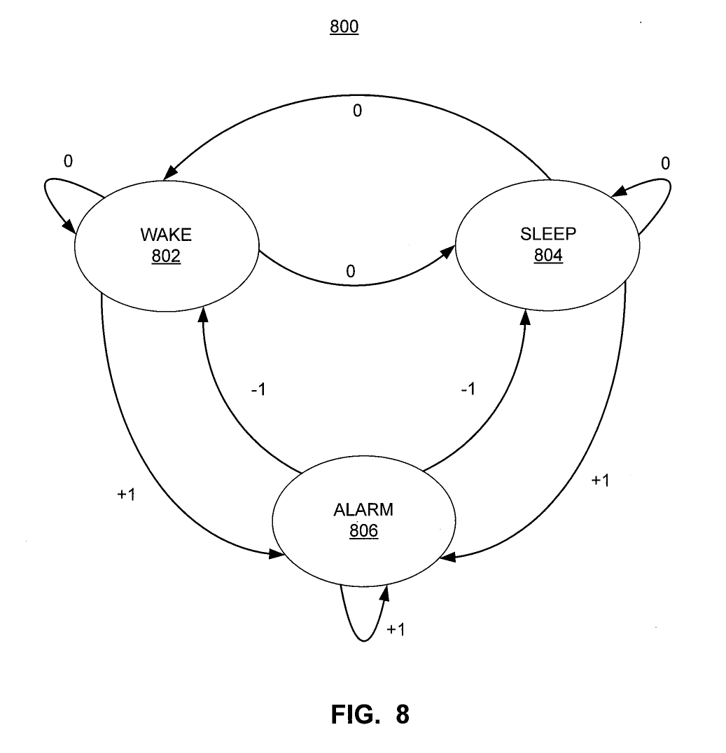

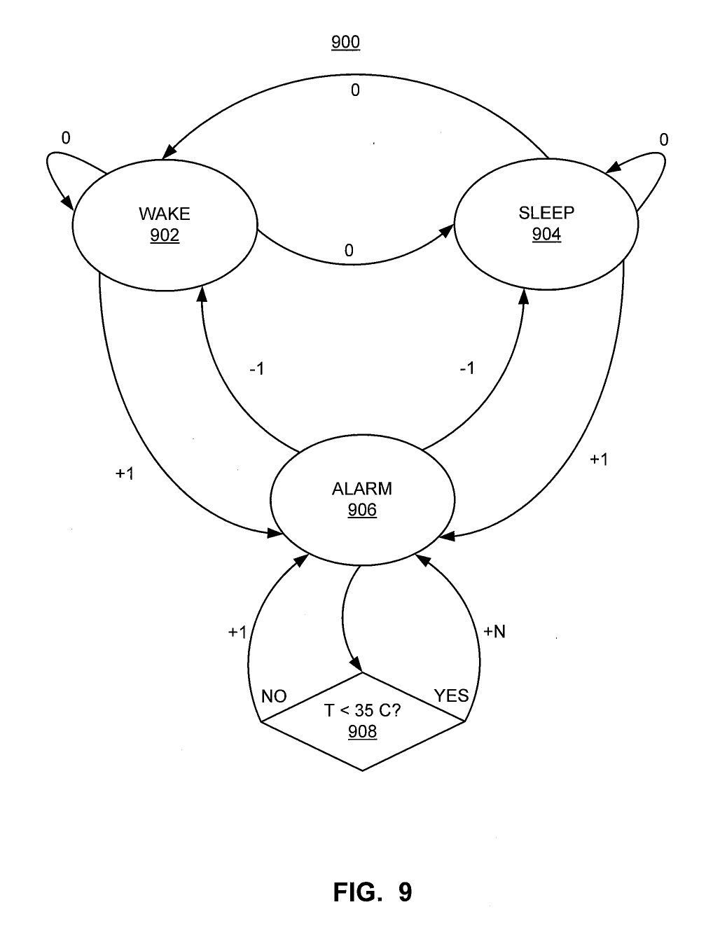

As represented by block 708, the monitoring process then involves determining a heart failure (e.g., heart failure exacerbation) score based on the newly collected information. In some implementations this score is updated based on a state transition algorithm. For example, a new score may be calculated as NEW SCORE=PRIOR SCORE+WEIGHT*(−1, 0, or +1), where the −1, 0, or +1 value is provided by the state transition algorithm. FIGS. 8 and 9 illustrate sample state transition diagrams representative of such an algorithm.

The state transition diagram 800 shown in FIG. 8 includes three states: a wake state 802, a sleep state 804, and an alarm state 806. A transition from the wake state 802 to the sleep state 804, and vice versa, results in a value of 0. Hence, if the prior set of temperature and LAP information corresponded to a wake or sleep state and the new set of temperature and LAP information corresponds to a wake or sleep state, the heart failure score is not changed at block 708.

A transition to the alarm state 806, on the other hand, results in a value of +1. Thus, if the prior set of temperature and LAP information corresponded to the alarm state 806, the wake state 802, or the sleep state 804 and the new set of temperature and LAP information corresponds to the alarm state 806, the heart failure score is increased by the WEIGHT at block 708.

A transition to the wake state 802 or the sleep state 804 from the alarm state 806 results in a value of −1. Thus, if the prior set of temperature and LAP information corresponded to the alarm state 806 and the new set of temperature and LAP information corresponds to the wake state 802 or the sleep state 804, the heart failure score is decreased by the WEIGHT at block 708.

Some type of filtering also may be employed to reset the heart failure score in some cases. For example, it is possible that the heart failure score may be at a non-zero value while the patient remains in either the wake state 802 and/or the sleep state 804 for a relatively long period of time (e.g., a week). In such a case, the heart failure score may be reset (e.g., to 0) after a defined period of time or after a defined number of state decisions.

FIG. 9 illustrates an example of a state transition algorithm that employs a temperature threshold. The wake state 902 and the sleep state 904 are similar to the wake state 802 and the sleep state 804, respectively. Here, however, if hypothermia is detected (e.g., the lowest detected temperature during the latest collection period is below 35 C) during the alarm state 906, the score is increased by a relatively large number (e.g., N=100) at block 908 so that medical assistance is immediately triggered. Thus, the alarm state 906 is effectively divided into a tiered alarm state based on temperature.

Referring again to FIG. 7, as represented by block 710, if the heart failure score is greater than a second threshold (e.g., corresponding to chronic heart failure), an urgent heart failure alarm message may be sent at block 712. For example, an implantable medical device may send a message via a wireless link to an external apparatus (e.g., a bedside monitoring system, a cell phone, and so on) where the message indicates that the patient is suffering from heart failure-induced hypothermia. The external apparatus may then send a message to a monitoring center (e.g., via a 911 call or other suitable message) that is able to summon emergency care for the patient.

The second threshold (threshold 2) may be defined in various ways. In some cases, this threshold comprises a defined quantity indicative of a certain number of recent occurrences of temperature and left atrial pressure information corresponding to the alarm state. For example, the threshold may correspond to an alarm state being active for more than 3 days within a given period of time (e.g., 4 consecutive days).

As represented by block 714, if the heart failure score is not greater than the second threshold but is greater than a first threshold (e.g., corresponding to acute heart failure), a heart failure exacerbation message (e.g., a text message) may be sent at block 716. For example, an implantable medical device may send a message to an external apparatus (e.g., a bedside monitoring system, a cell phone, and so on) to indicate to the patient or a medical provider that a change in the therapy regimen may be needed. For example, the message may indicate that the dosage of the patient's medication (e.g., diuretic) needs to be increased. In the event the regimen change is effective, subsequent iterations of the process of FIG. 7 may result in a non-alarm state which will cause the heart failure score to decrease.

The first threshold (threshold 1) also may be defined in various ways. In some cases, this threshold comprises a defined quantity indicative of a certain number of recent occurrences of temperature and left atrial pressure information corresponding to the alarm state. For example, the threshold may correspond to an alarm state being active for more than 1 day within a given period of time (e.g., 2 consecutive days).

Referring now to FIGS. 10-12, the disclosure relates in some aspects to monitoring heart failure status based on a perfusion/congestion (warm-cold/wet-dry) diagnostic trend diagram. For example, in accordance with the teachings herein, a 2-D graph based on collected perfusion and congestion information may be displayed for a treating physician to enable the physician to monitor chronological heart failure status.

FIG. 10 illustrates sample operations that may be performed to provide a 2-D indication of a heart failure trend based on congestion information and perfusion information collected from a patient. In some aspects, the congestion information provides an indication of lung fluid congestion in a patient and the perfusion information provides an indication of peripheral circulation in the patient. In some aspects, a higher degree of congestion correlates to higher left atrial pressure, and vice versa. In addition, in some aspects, a lower degree of perfusion correlates to lower dP/dt (e.g., rate of change of blood pressure in a vessel such as a ventricle).

As represented by blocks 1002 and 1004, congestion information and perfusion information are collected over time. For example, an implantable medical device may employ sensing circuits and/or may be coupled to other sensing circuits (e.g., implantable sensing circuits) that sense signals from which congestion information and perfusion information may be derived.

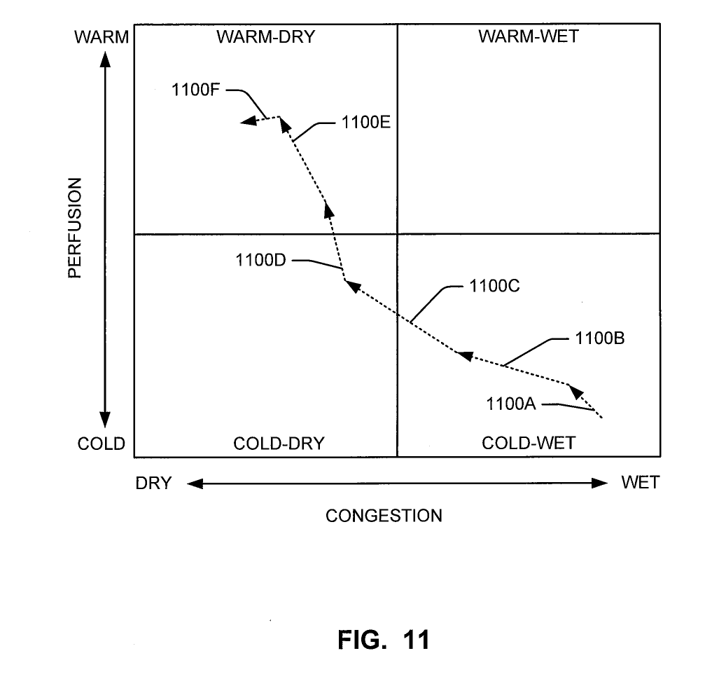

As represented by block 1006, a 2-D indication of a heart failure trend may then be provided based on the information collected at blocks 1002 and 1004. FIG. 11 depicts a sample 2-D indication of a heart failure trend.

As shown in FIG. 11, a heart failure trend may be advantageously presented with respect to a warm-cold/wet-dry heart failure classification diagram as described in Grady et al., “Team Management of Patients With Heart Failure: A Statement for Healthcare Professionals From the Cardiovascular Nursing Council of the American Heart Association,” Circulation, 2000; 102:2443-2456. Here, adequate perfusion may be characterized as a warm profile while a compromise of tissue perfusion (critical hypoperfusion) may be characterized as a cold profile. Also, the presence of congestion may be characterized as a wet profile while the absence of congestion may be characterized as a dry profile.

The Brady et al. article also identifies treatment regimes for each classification. For example, for a warm and dry classification (e.g., an optimal profile), a treating physician may focus on prevention of disease progression and decompensation. For a wet and warm classification, diuresis with continuation of standard therapy may be prescribed. For a cold and dry classification, there may be limited therapy options. For a wet and cold classification, diuresis and a redesign of the regimen with other standard therapies may be prescribed.

By providing detailed trend (e.g., trajectory) information on such a graph as taught herein (e.g., as opposed to simply categorizing a patient's current state as one of the four classifications), a treating physician may advantageously be able to monitor the patient's past heart failure status, monitor heart failure progression, determine whether a particular regimen is effective, and thereby determine how and/or when to modify a regimen for the patient. For example, the physician may use the 2-D graph to examine the effects of drug titration and interpret a trend trajectory to determine when to decrease a medication dosage to avoid a “too dry” situation. Also, the physician may interpret a trend trajectory to determine how and/or when to adjust device parameters (e.g., stimulation parameters of an implantable cardiac stimulation device) and assess the degree of peripheral vaso-dilation.

Moreover, by acquiring congestion and perfusion information via sensors and physiological parameters as taught herein, such a graph may provide congestion and perfusion information at a high level of sensitivity. For example, sensing circuits (e.g., sensors) used to provide this information may employ pulmonary edema (PE) sensing, cardiogenic impedance (CI) sensing, evoked response (ER) morphology sensing, electrical delay sensing, exercise compliance sensing, peripheral temperature sensing, body core temperature sensing, and photoplethysmography-based sensing.

The trend data of FIG. 11 describes a patient's heart failure status over a period of time (e.g., several weeks). For example, the trend may commence at the beginning of arrow 1100A with data collection on a certain day. For example, the patient may have started a new treatment regimen on this day.

The end of the arrow 1100A (and the beginning of the arrow 1100B) may correspond to a later point in time (e.g., one week later), whereby improvement in the patient's condition over that period of time is indicated. Based on this improvement, the treating physician may, for example, elect to bolster the prescribed regimen (e.g., increase the dosage of medication).

The end of the arrow 1100B (and the beginning of the arrow 1100C) may correspond to a still later point in time (e.g., another week later), whereby additional improvement in the patient's condition over that period of time is indicated. Based on this trend information, the treating physician may, for example, elect to maintain the current regimen.

The end of the arrow 1100C (and the beginning of the arrow 1100D) may correspond to a later point in time (e.g., yet another week later), whereby improvement in the patient's condition is indicated (e.g., the patient's heart failure status has moved to the cold-dry classification). Based on this status, the treating physician may, for example, elect to change the prescribed regimen.

The end of the arrow 1100D (and the beginning of the arrow 1100E) may correspond to a later point in time (e.g., another week later), whereby further improvement in the patient's condition is indicated. Based on this trend information, the treating physician may, for example, elect to maintain the current regimen.

The end of the arrow 1100E (and the beginning of the arrow 1100F) may correspond to a later point in time (e.g., yet another week later), whereby improvement in the patient's condition is indicated (e.g., the patient's heart failure status is well within the warm-dry classification). Based on this status, the treating physician may, for example, elect to change the prescribed regimen again (e.g., to simply maintain the patient within the warm-dry classification).

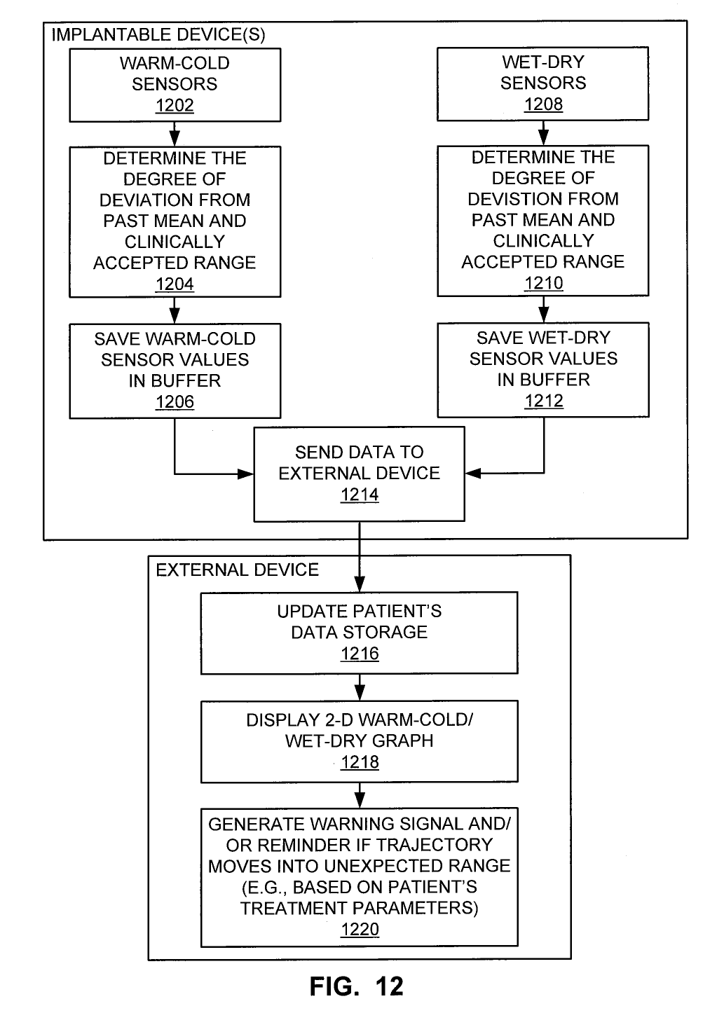

FIG. 12 illustrates in more detail a sample monitoring process that provides a 2-D heart failure status indication in accordance with the teachings herein. In general, this process may be performed on a repeated basis (e.g., hourly, twice a day, once a day, or at other times). However, this monitoring schedule may be dynamically changed based on a defined trigger event. For example, in the event heart failure exacerbation is detected, the monitoring may be performed more frequently (e.g., hourly, every two hours, etc.).

In the example, of FIG. 12, different operations are performed by an implantable device (or devices) and an external device. Specifically, operations 1202-1214 relating to acquiring congestion (wet-dry) information and perfusion (warm-cold) information are performed by one or more devices implanted in a patient. In addition, a device located external to the patient performs operations relating to providing indications based on the congestion and perfusion information.

As represented by blocks 1202 and 1208, an implantable monitoring device may employ sensing circuits (e.g., sensors) and/or may be coupled to other sensing circuits to acquire congestion and perfusion information. As an example of the latter case, a sensing circuit may be implanted in a patient and coupled to the implanted monitoring device via an implantable lead or via a wireless communication link.

A perfusion (warm-cold) sensing circuit may take various forms. In some implementations a perfusion sensing circuit comprises one or more temperature sensors (e.g., for measuring core temperature and/or peripheral temperature). Here, a decrease in temperature corresponds to a decrease in perfusion. In some implementations a perfusion sensing circuit may measure cardiogenic impedance (CI). For example, a decrease in cardiogenic impedance may correspond to a decrease in perfusion. In some implementations a perfusion sensing circuit may sense arteriole stiffness. For example, a photoplethysmography (PPG) sensor may be used sense pressure waves in a cardiac vessel to determine how pressure waves are reflected by arteriole vessel walls or other cardiac vessel walls. Here, an increase in arteriole stiffness (corresponding to a decrease in perfusion) may be indicated by a faster than normal reflection time for the pressure wave.

A congestion (wet-dry) sensing circuit may take various forms. In some implementations a congestion sensing circuit comprises a pulmonary edema sensing circuit. For example, impedance changes may be measured by sensing for changes in voltage that result from the application of a constant current between an implanted monitoring device (e.g., implantable cardiac stimulation device) and an implanted cardiac lead (e.g., a left ventricle tip electrode). Here, a lower than normal resistance may indicate a pulmonary edema condition (corresponding to congestion). In some implementations a congestion sensing circuit comprises an evoked response sensing circuit. For example, an implantable cardiac stimulation device may monitor evoked response signals (e.g., cardiac signals generated in response to a pacing pulse) through the use of electrodes on implanted cardiac leads. Here, increased congestion may be indicated by, for example, a lengthening of the duration of a segment of an evoked response signal, a reduction in the post depolarization integral (PDI), or a reduction in the slope of an evoked response signal.

As represented by blocks 1204 and 1210, the implantable monitoring device may determine the extent (e.g., severity) to which the information acquired by blocks 1202 and 1208 deviates from a previously determined mean and/or from a clinically accepted range. This may involve, for example, determining a delta for the trend and determining whether an indication (e.g., an alarm) needs to be generated as a result of detection of particularly critical perfusion or congestion values. For example, a temperature below 35 C or a blood pressure reading above 15 mmHg may cause the implantable monitoring device to generate an indication that an alarm signal is to be generated (e.g., at block 1220 as discussed below).

As represented by blocks 1206 and 1212, the implantable monitoring device may store the perfusion and congestion information in a memory device (e.g., a buffer). The implantable monitoring device may then send this information to the external device as represented by block 1214. For example, updated trend information may be sent to the external device via a wireless link each time new congestion and perfusion information is collected by the implantable monitoring device (e.g., on a daily basis).

As represented by block 1216, the external device collects the most recent trend information sent by the patient's implantable monitoring device and updates the information the external device maintains pertaining to that patient. Accordingly, the external device may maintain chronological trend information indicative of the congestion and perfusion status of the patient at various points in time over a period of time.

As represented by block 1218, the external device may then provide a 2-D graph of the congestion/perfusion trend. For example, the external device may display a graph similar to FIG. 11 on a display device.

As represented by block 1220, the external device also may generate an indication (e.g., a warning signal or reminder) if certain defined conditions relating to the trend information are met. For example, such an indication may be generated if the trajectory moves into an unexpected or undesirable range (e.g., temperature below 35 C or blood pressure above 15 mmHg). Here, the defined conditions may be patient-specific (e.g., based on the patient's treatment parameters).

In some implementations, the external device may send the trend information to another entity that then provides one or more indications based on the trend information. For example, the external device may send the trend information to a monitoring center. Then monitoring center may then display the 2-D congestion/profusion graph and generate any other indications (e.g., warnings or reminders) that are warranted. Thus, in some implementations, the monitoring center may perform operations similar to blocks 1216-1220.

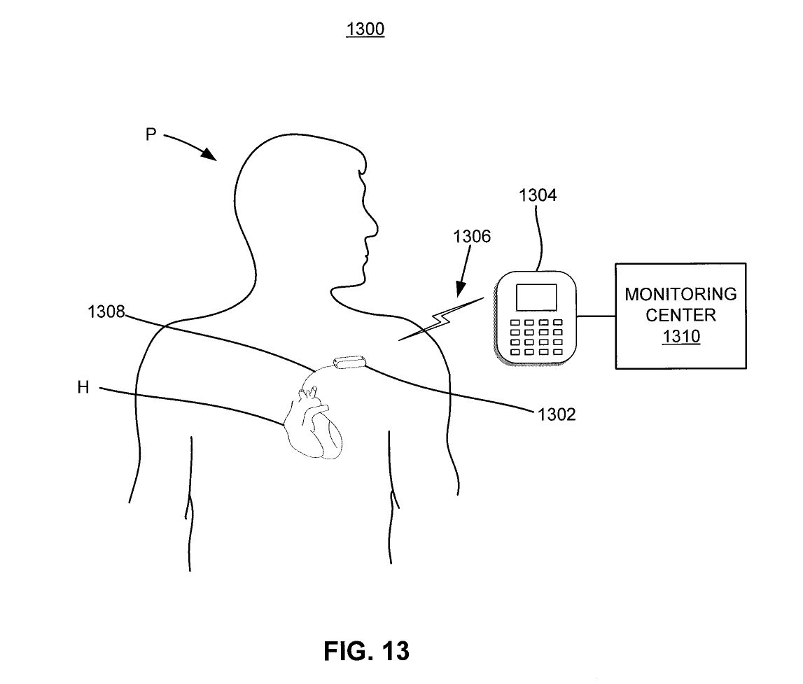

FIG. 13 illustrates a simplified diagram of a device 1302 (implanted within a patient P) that communicates with a device 1304 that is located external to the patient P. The implanted device 1302 and the external device 1304 may communicate with one another via a wireless communication link 1306 (as represented by the depicted wireless symbol).

In the illustrated example, the implanted device 1302 is an implantable cardiac device including one or more leads 1308 that are routed to the heart H of the patient P. For example, the implanted device 1302 may be a pacemaker, an implantable cardioverter defibrillator, or some other similar device. It should be appreciated, however, that the implanted device 1302 may take other forms.

The external device 1304 also may take various forms. For example, the external device 1304 may be a base station, a programmer, a home safety monitor, a personal monitor, a follow-up monitor, a wearable monitor, or some other type of device that is configured to communicate with the implanted device 1302.

The communication link 1306 may be used to transfer information between the devices 1302 and 1304 in conjunction with various applications such as remote home-monitoring, clinical visits, data acquisition, remote follow-up, and portable or wearable patient monitoring/control systems. For example, information (e.g., LAP, temperature, congestion, or perfusion information) may be transferred between the devices 1302 and 1304 when the patient P is at a location that is relatively close to the external device 1304. Here, information transfers may be invoked upon command, at designated times, or in some other manner.

As discussed above in conjunction with FIG. 13, an external device may send information it receives from an implanted device to another device (e.g., that may provide a more convenient means for a physician to review the information). For example, the external device 1304 may send the information to a monitoring center 1310 (e.g., via a web server). In this way, monitoring personnel (e.g., a physician) may remotely access the information (e.g., by accessing a website). The monitoring personnel may then review the information uploaded from the implantable device to determine whether medical intervention is warranted.

The information acquisition operations and indication generation operations discussed herein may be implemented in an implantable device and/or an external device in different implementations. In some implementations an implantable device may include a heart failure information acquisition circuit that collects information (e.g., by performing operations corresponding to one or more of blocks 302, 304, 702, 704, 1002, 1004, or 1202-1212 described above) and an indication generator that provides, for example, an audible or vibratory alarm or sends messages (e.g., by performing operations corresponding to one or more of blocks 306-310, 706-716, 1006, 1214, or 1220 described above).

In some implementations an implantable device includes a heart failure information acquisition circuit and is configured to send the acquired (e.g., collected) information to an external device. In these implementations an external device (e.g., the external device 1304 or a device at the monitoring center 1310) may include a heart failure information acquisition circuit (e.g., that collects information received from an implantable device, an external device, or in some other manner) and an indication generator (e.g., that sends messages or provides a visual display or an audible alarm). Thus, in various implementations, such a heart failure information acquisition circuit may perform operations corresponding to one or more of blocks 302, 304, 702, 704, 1002, 1004, or 1202-1212 described above and an indication generator may perform operations corresponding to one or more of blocks 306-310, 706-716, 1006, 1214, 1218, or 1220 described above.

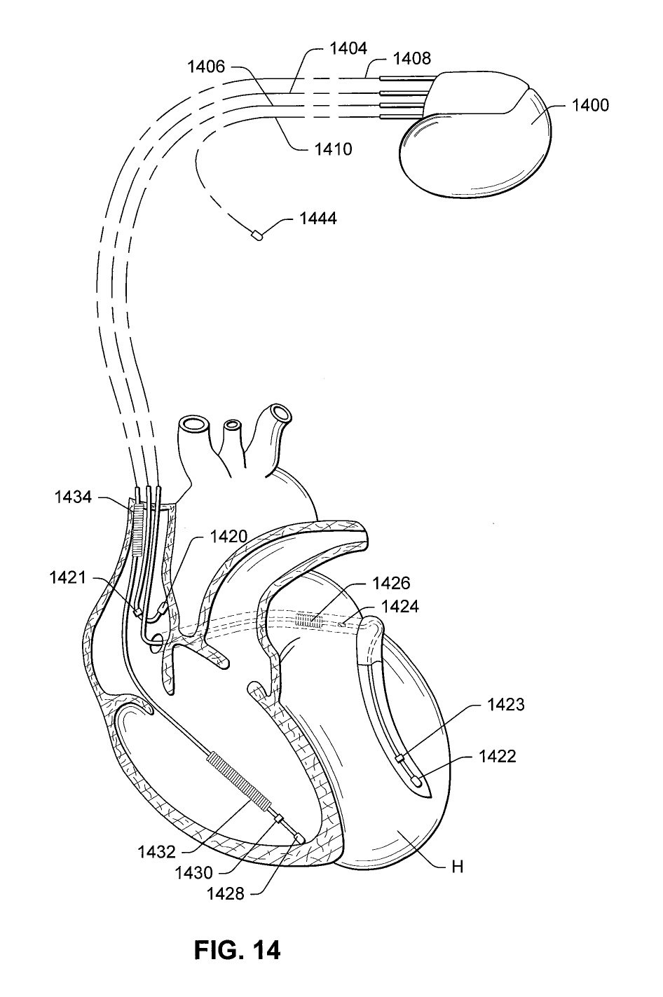

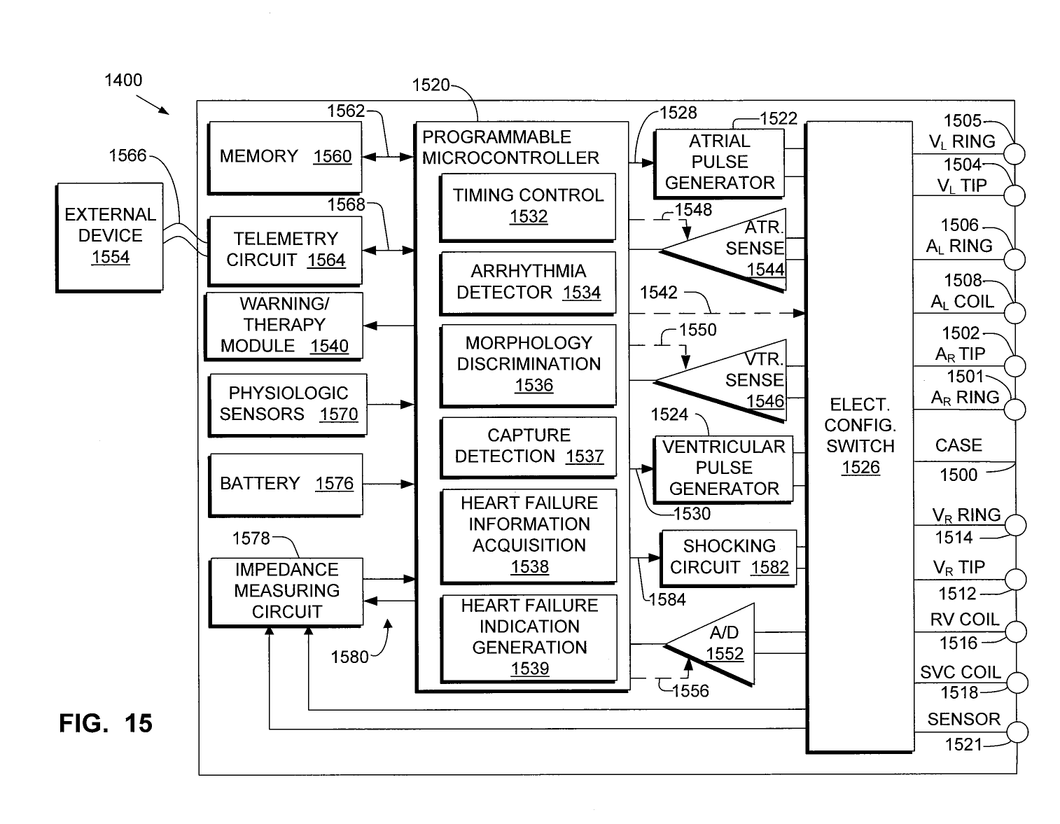

Referring now to FIGS. 14 and 15, an example of an implantable cardiac device (e.g., a stimulation device such as an implantable cardioverter defibrillator, a pacemaker, etc.) that may be configured to provide monitoring in accordance with the teachings herein will be described. It is to be appreciated and understood that other cardiac devices, including those that are not necessarily implantable, may be used and that the description below is given, in its specific context, to assist the reader in understanding, with more clarity, sample uses of the embodiments described herein.

FIG. 14 shows an exemplary implantable cardiac device 1400 in electrical communication with a patient's heart H by way of three leads 1404, 1406, and 1408, suitable for delivering multi-chamber stimulation and shock therapy. Bodies of the leads 1404, 1406, and 1408 may be formed of silicone, polyurethane, plastic, or similar biocompatible materials to facilitate implant within a patient. Each lead includes one or more conductors, each of which may couple one or more electrodes incorporated into the lead to a connector on the proximal end of the lead. Each connector, in turn, is configured to couple with a complimentary connector (e.g., implemented within a header) of the device 1400.

To sense atrial cardiac signals and to provide right atrial chamber stimulation therapy, the device 1400 is coupled to an implantable right atrial lead 1404 having, for example, an atrial tip electrode 1420, which typically is implanted in the patient's right atrial appendage or septum. FIG. 14 also shows the right atrial lead 1404 as having an optional atrial ring electrode 1421.

To sense left atrial and ventricular cardiac signals and to provide left chamber pacing therapy, the device 1400 is coupled to a coronary sinus lead 1406 designed for placement in the coronary sinus region via the coronary sinus for positioning one or more electrodes adjacent to the left ventricle, one or more electrodes adjacent to the left atrium, or both. As used herein, the phrase “coronary sinus region” refers to the vasculature of the left ventricle, including any portion of the coronary sinus, the great cardiac vein, the left marginal vein, the left posterior ventricular vein, the middle cardiac vein, the small cardiac vein or any other cardiac vein accessible by the coronary sinus.

Accordingly, an exemplary coronary sinus lead 1406 is designed to receive atrial and ventricular cardiac signals and to deliver left ventricular pacing therapy using, for example, a left ventricular tip electrode 1422 and, optionally, a left ventricular ring electrode 1423; provide left atrial pacing therapy using, for example, a left atrial ring electrode 1424; and provide shocking therapy using, for example, a left atrial coil electrode 1426 (or other electrode capable of delivering a shock). For a more detailed description of a coronary sinus lead, the reader is directed to U.S. Pat. No. 5,466,254, “Coronary Sinus Lead with Atrial Sensing Capability” (Helland), which is incorporated herein by reference.

The device 1400 is also shown in electrical communication with the patient's heart H by way of an implantable right ventricular lead 1408 having, in this implementation, a right ventricular tip electrode 1428, a right ventricular ring electrode 1430, a right ventricular (RV) coil electrode 1432 (or other electrode capable of delivering a shock), and a superior vena cava (SVC) coil electrode 1434 (or other electrode capable of delivering a shock). Typically, the right ventricular lead 1408 is transvenously inserted into the heart H to place the right ventricular tip electrode 1428 in the right ventricular apex so that the RV coil electrode 1432 will be positioned in the right ventricle and the SVC coil electrode 1434 will be positioned in the superior vena cava. Accordingly, the right ventricular lead 1408 is capable of sensing or receiving cardiac signals, and delivering stimulation in the form of pacing and shock therapy to the right ventricle.

The device 1400 is also shown in electrical communication with a lead 1410 including one or more components 1444 such as, for example, a physiologic sensor or some other type of sensing circuit. For example, a component 1444 may comprise one or more of: circuitry for sensing LAP, circuitry for sensing temperature, circuitry for sensing congestion, circuitry for sensing perfusion, or some other type of sensing circuitry. A component 1444 may be positioned in, near or remote from the heart.

It should be appreciated that the device 1400 may connect to leads other than those specifically shown. In addition, the leads connected to the device 1400 may include components other than those specifically shown. For example, a lead may include other types of electrodes, sensors or devices that serve to otherwise interact with a patient or the surroundings.

FIG. 15 depicts an exemplary, simplified block diagram illustrating sample components of the device 1400. The device 1400 may be adapted to treat both fast and slow arrhythmias with stimulation therapy, including cardioversion, defibrillation, and pacing stimulation. While a particular multi-chamber device is shown, it is to be appreciated and understood that this is done for illustration purposes. Thus, the techniques and methods described below can be implemented in connection with any suitably configured or configurable device. Accordingly, one of skill in the art could readily duplicate, eliminate, or disable the appropriate circuitry in any desired combination to provide a device capable of treating the appropriate chamber(s) with, for example, cardioversion, defibrillation, and pacing stimulation.

A housing 1500 for the device 1400 is often referred to as the “can”, “case” or “case electrode”, and may be programmably selected to act as the return electrode for all “unipolar” modes. The housing 1500 may further be used as a return electrode alone or in combination with one or more of the coil electrodes 1426, 1432 and 1434 for shocking purposes. The housing 1500 may be constructed of a biocompatible material (e.g., titanium) to facilitate implant within a patient.

The housing 1500 further includes a connector (not shown) having a plurality of terminals 1501, 1502, 1504, 1505, 1506, 1508, 1512, 1514, 1516 and 1518 (shown schematically and, for convenience, the names of the electrodes to which they are connected are shown next to the terminals). The connector may be configured to include various other terminals depending on the requirements of a given application. For example, terminal(s) 1521 may be coupled to one or more of: circuitry for sensing LAP, circuitry for sensing temperature, circuitry for sensing congestion, circuitry for sensing perfusion, or some other type of sensing component.

To achieve right atrial sensing and pacing, the connector includes, for example, a right atrial tip terminal (AR TIP) 1502 adapted for connection to the right atrial tip electrode 1420. A right atrial ring terminal (AR RING) 1501 may also be included and adapted for connection to the right atrial ring electrode 1421. To achieve left chamber sensing, pacing, and shocking, the connector includes, for example, a left ventricular tip terminal (VL TIP) 1504, a left ventricular ring terminal (VL RING) 1505, a left atrial ring terminal (AL RING) 1506, and a left atrial shocking terminal (AL COIL) 1508, which are adapted for connection to the left ventricular tip electrode 1422, the left ventricular ring electrode 1423, the left atrial ring electrode 1424, and the left atrial coil electrode 1426, respectively.

To support right chamber sensing, pacing, and shocking, the connector further includes a right ventricular tip terminal (VR TIP) 1512, a right ventricular ring terminal (VR RING) 1514, a right ventricular shocking terminal (RV COIL) 1516, and a superior vena cava shocking terminal (SVC COIL) 1518, which are adapted for connection to the right ventricular tip electrode 1428, the right ventricular ring electrode 1430, the RV coil electrode 1432, and the SVC coil electrode 1434, respectively.

At the core of the device 1400 is a programmable microcontroller 1520 that controls the various modes of stimulation therapy. As is well known in the art, microcontroller 1520 typically includes a microprocessor, or equivalent control circuitry, designed specifically for controlling the delivery of stimulation therapy, and may further include memory such as RAM, ROM and flash memory, logic and timing circuitry, state machine circuitry, and I/O circuitry. Typically, microcontroller 1520 includes the ability to process or monitor input signals (data or information) as controlled by a program code stored in a designated block of memory. The type of microcontroller is not critical to the described implementations. Rather, any suitable microcontroller 1520 may be used that carries out the functions described herein. The use of microprocessor-based control circuits for performing timing and data analysis functions are well known in the art.

Representative types of control circuitry that may be used in connection with the described embodiments can include the microprocessor-based control system of U.S. Pat. No. 4,940,052 (Mann et al.), the state-machine of U.S. Pat. Nos. 4,712,555 (Thornander et al.) and 4,944,298 (Sholder), all of which are incorporated by reference herein. For a more detailed description of the various timing intervals that may be used within the device and their inter-relationship, see U.S. Pat. No. 4,788,980 (Mann et al.), also incorporated herein by reference.

FIG. 15 also shows an atrial pulse generator 1522 and a ventricular pulse generator 1524 that generate pacing stimulation pulses for delivery by the right atrial lead 1404, the coronary sinus lead 1406, the right ventricular lead 1408, or some combination of these leads via an electrode configuration switch 1526. It is understood that in order to provide stimulation therapy in each of the four chambers of the heart, the atrial and ventricular pulse generators 1522 and 1524 may include dedicated, independent pulse generators, multiplexed pulse generators, or shared pulse generators. The pulse generators 1522 and 1524 are controlled by the microcontroller 1520 via appropriate control signals 1528 and 1530, respectively, to trigger or inhibit the stimulation pulses.

Microcontroller 1520 further includes timing control circuitry 1532 to control the timing of the stimulation pulses (e.g., pacing rate, atrio-ventricular (A-V) delay, atrial interconduction (A-A) delay, or ventricular interconduction (V-V) delay, etc.) or other operations, as well as to keep track of the timing of refractory periods, blanking intervals, noise detection windows, evoked response windows, alert intervals, marker channel timing, etc., as known in the art.

Microcontroller 1520 further includes an arrhythmia detector 1534. The arrhythmia detector 1534 may be utilized by the device 1400 for determining desirable times to administer various therapies. The arrhythmia detector 1534 may be implemented, for example, in hardware as part of the microcontroller 1520, or as software/firmware instructions programmed into the device 1400 and executed on the microcontroller 1520 during certain modes of operation.

Microcontroller 1520 may include a morphology discrimination module 1536, a capture detection module 1537 and an auto sensing module (not shown). These modules are optionally used to implement various exemplary recognition algorithms or methods. The aforementioned components may be implemented, for example, in hardware as part of the microcontroller 1520, or as software/firmware instructions programmed into the device 1400 and executed on the microcontroller 1520 during certain modes of operation.

The electrode configuration switch 1526 includes a plurality of switches for connecting the desired terminals (e.g., that are connected to electrodes, coils, sensors, etc.) to the appropriate I/O circuits, thereby providing complete terminal and, hence, electrode programmability. Accordingly, switch 1526, in response to a control signal 1542 from the microcontroller 1520, may be used to determine the polarity of the stimulation pulses (e.g., unipolar, bipolar, combipolar, etc.) by selectively closing the appropriate combination of switches (not shown) as is known in the art.

Atrial sensing circuits 1544 and ventricular sensing circuits 1546 may also be selectively coupled to the right atrial lead 1404, coronary sinus lead 1406, and the right ventricular lead 1408, through the switch 1526 for detecting the presence of cardiac activity in each of the four chambers of the heart. Accordingly, the atrial and ventricular sensing circuits 1544 and 1546 may include dedicated sense amplifiers, multiplexed amplifiers, or shared amplifiers. Switch 1526 determines the “sensing polarity” of the cardiac signal by selectively closing the appropriate switches, as is also known in the art. In this way, the clinician may program the sensing polarity independent of the stimulation polarity. The sensing circuits (e.g., circuits 1544 and 1546) are optionally capable of obtaining information indicative of tissue capture.

Each sensing circuit 1544 and 1546 preferably employs one or more low power, precision amplifiers with programmable gain, automatic gain control, bandpass filtering, a threshold detection circuit, or some combination of these components, to selectively sense the cardiac signal of interest. The automatic gain control enables the device 1400 to deal effectively with the difficult problem of sensing the low amplitude signal characteristics of atrial or ventricular fibrillation.

The outputs of the atrial and ventricular sensing circuits 1544 and 1546 are connected to the microcontroller 1520, which, in turn, is able to trigger or inhibit the atrial and ventricular pulse generators 1522 and 1524, respectively, in a demand fashion in response to the absence or presence of cardiac activity in the appropriate chambers of the heart. Furthermore, as described herein, the microcontroller 1520 is also capable of analyzing information output from the sensing circuits 1544 and 1546, a data acquisition system 1552, or both. This information may be used to determine or detect whether and to what degree tissue capture has occurred and to program a pulse, or pulses, in response to such determinations. The sensing circuits 1544 and 1546, in turn, receive control signals over signal lines 1548 and 1550, respectively, from the microcontroller 1520 for purposes of controlling the gain, threshold, polarization charge removal circuitry (not shown), and the timing of any blocking circuitry (not shown) coupled to the inputs of the sensing circuits 1544 and 1546 as is known in the art.

For arrhythmia detection, the device 1400 utilizes the atrial and ventricular sensing circuits 1544 and 1546 to sense cardiac signals to determine whether a rhythm is physiologic or pathologic. It should be appreciated that other components may be used to detect arrhythmia depending on the system objectives. In reference to arrhythmias, as used herein, “sensing” is reserved for the noting of an electrical signal or obtaining data (information), and “detection” is the processing (analysis) of these sensed signals and noting the presence of an arrhythmia.

Timing intervals between sensed events (e.g., P-waves, R-waves, and depolarization signals associated with fibrillation) may be classified by the arrhythmia detector 1534 of the microcontroller 1520 by comparing them to a predefined rate zone limit (e.g., bradycardia, normal, low rate VT, high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”). Similar rules may be applied to the atrial channel to determine if there is an atrial tachyarrhythmia or atrial fibrillation with appropriate classification and intervention.

Cardiac signals or other signals may be applied to inputs of an analog-to-digital (ND) data acquisition system 1552. The data acquisition system 1552 is configured (e.g., via signal line 1556) to acquire intracardiac electrogram (“IEGM”) signals or other signals, convert the raw analog data into a digital signal, and store the digital signals for later processing, for telemetric transmission to an external device 1554, or both. For example, the data acquisition system 1552 may be coupled to the right atrial lead 1404, the coronary sinus lead 1406, the right ventricular lead 1408 and other leads through the switch 1526 to sample cardiac signals across any pair of desired electrodes.

The data acquisition system 1552 also may be coupled to receive signals from other input devices. For example, the data acquisition system 1552 may sample signals from a physiologic sensor 1570 or other components shown in FIG. 15 (connections not shown).

The microcontroller 1520 is further coupled to a memory 1560 by a suitable data/address bus 1562, wherein the programmable operating parameters used by the microcontroller 1520 are stored and modified, as required, in order to customize the operation of the device 1400 to suit the needs of a particular patient. Such operating parameters define, for example, pacing pulse amplitude, pulse duration, electrode polarity, rate, sensitivity, automatic features, arrhythmia detection criteria, and the amplitude, waveshape and vector of each shocking pulse to be delivered to the patient's heart H within each respective tier of therapy. One feature of the described embodiments is the ability to sense and store a relatively large amount of data (e.g., from the data acquisition system 1552), which data may then be used for subsequent analysis to guide the programming of the device 1400.

Advantageously, the operating parameters of the implantable device 1400 may be non-invasively programmed into the memory 1560 through a telemetry circuit 1564 in telemetric communication via communication link 1566 with the external device 1554, such as a programmer, transtelephonic transceiver, a diagnostic system analyzer or some other device. The microcontroller 1520 activates the telemetry circuit 1564 with a control signal (e.g., via bus 1568). The telemetry circuit 1564 advantageously allows intracardiac electrograms and status information relating to the operation of the device 1400 (as contained in the microcontroller 1520 or memory 1560) to be sent to the external device 1554 through an established communication link 1566.

The device 1400 can further include one or more physiologic sensors 1570. In some embodiments the device 1400 may include a “rate-responsive” sensor that may provide, for example, information to aid in adjustment of pacing stimulation rate according to the exercise state of the patient. One or more physiologic sensors 1570 (e.g., a pressure sensor) may further be used to detect changes in cardiac output, changes in the physiological condition of the heart, or diurnal changes in activity (e.g., detecting sleep and wake states). Accordingly, the microcontroller 1520 responds by adjusting the various pacing parameters (such as rate, A-V Delay, V-V Delay, etc.) at which the atrial and ventricular pulse generators 1522 and 1524 generate stimulation pulses. In various implementations the physiologic sensor 1570 may represent one or more of: an LAP sensor, a temperature sensor, circuitry for sensing congestion, or circuitry for sensing perfusion.

While shown as being included within the device 1400, it is to be understood that a physiologic sensor 1570 may also be external to the device 1400, yet still be implanted within or carried by the patient. Examples of physiologic sensors that may be implemented in conjunction with the device 1400 include sensors that sense respiration rate, pH of blood, ventricular gradient, oxygen saturation, blood pressure and so forth. Another sensor that may be used is one that detects activity variance, wherein an activity sensor is monitored diurnally to detect the low variance in the measurement corresponding to the sleep state. For a more detailed description of an activity variance sensor, the reader is directed to U.S. Pat. No. 5,476,483 (Bornzin et al.), which patent is hereby incorporated by reference.

The one or more physiologic sensors 1570 may optionally include one or more of components to help detect movement (via, e.g., a position sensor or an accelerometer) and minute ventilation (via an MV sensor) in the patient. Signals generated by the position sensor and MV sensor may be passed to the microcontroller 1520 for analysis in determining whether to adjust the pacing rate, etc. The microcontroller 1520 may thus monitor the signals for indications of the patient's position and activity status, such as whether the patient is climbing up stairs or descending down stairs or whether the patient is sitting up after lying down.

The device 1400 additionally includes a battery 1576 that provides operating power to all of the circuits shown in FIG. 15. For a device 1400 which employs shocking therapy, the battery 1576 is capable of operating at low current drains (e.g., preferably less than 10 μA) for long periods of time, and is capable of providing high-current pulses (for capacitor charging) when the patient requires a shock pulse (e.g., preferably, in excess of 2 A, at voltages above 200 V, for periods of 10 seconds or more). The battery 1576 also desirably has a predictable discharge characteristic so that elective replacement time can be detected. Accordingly, the device 1400 preferably employs lithium or other suitable battery technology.

The device 1400 can further include magnet detection circuitry (not shown), coupled to the microcontroller 1520, to detect when a magnet is placed over the device 1400. A magnet may be used by a clinician to perform various test functions of the device 1400 and to signal the microcontroller 1520 that the external device 1554 is in place to receive data from or transmit data to the microcontroller 1520 through the telemetry circuit 1564.

The device 1400 further includes an impedance measuring circuit 1578 that is enabled by the microcontroller 1520 via a control signal 1580. The known uses for an impedance measuring circuit 1578 include, but are not limited to, lead impedance surveillance during the acute and chronic phases for proper performance, lead positioning or dislodgement; detecting operable electrodes and automatically switching to an operable pair if dislodgement occurs; measuring respiration or minute ventilation; measuring thoracic impedance for determining shock thresholds; detecting when the device 1400 has been implanted; measuring stroke volume; and detecting the opening of heart valves, etc. The impedance measuring circuit 1578 is advantageously coupled to the switch 1526 so that any desired electrode may be used.

In the case where the device 1400 is intended to operate as an implantable cardioverter/defibrillator (ICD) device, it detects the occurrence of an arrhythmia, and automatically applies an appropriate therapy to the heart aimed at terminating the detected arrhythmia. To this end, the microcontroller 1520 further controls a shocking circuit 1582 by way of a control signal 1584. The shocking circuit 1582 generates shocking pulses of low (e.g., up to 0.5 J), moderate (e.g., 0.5 J to 10 J), or high energy (e.g., 11 J to 40 J), as controlled by the microcontroller 1520. Such shocking pulses are applied to the patient's heart H through, for example, two shocking electrodes and as shown in this embodiment, selected from the left atrial coil electrode 1426, the RV coil electrode 1432 and the SVC coil electrode 1434. As noted above, the housing 1500 may act as an active electrode in combination with the RV coil electrode 1432, as part of a split electrical vector using the SVC coil electrode 1434 or the left atrial coil electrode 1426 (i.e., using the RV electrode as a common electrode), or in some other arrangement.

Cardioversion level shocks are generally considered to be of low to moderate energy level (so as to minimize pain felt by the patient), be synchronized with an R-wave, pertain to the treatment of tachycardia, or some combination of the above. Defibrillation shocks are generally of moderate to high energy level (i.e., corresponding to thresholds in the range of 5 J to 40 J), delivered asynchronously (since R-waves may be too disorganized), and pertaining to the treatment of fibrillation. Accordingly, the microcontroller 1520 is capable of controlling the synchronous or asynchronous delivery of the shocking pulses.

As mentioned above, the device 1400 may include several components that provide the monitoring functionality as taught herein. For example, one or more of the switch 1526, the sense circuits 1544, 1546, and the data acquisition system 1552 may acquire signals that are used in the monitoring operations discussed above, with reference to FIGS. 3-7 and 10-12. The collected data described above may be stored in the memory 1560. In addition, a warning/therapy module 1540 may be configured to generate warning signals upon detection of heart failure exacerbation and, in the case of an implantable stimulation device, facilitate the administration of therapy.

The microcontroller 1520 (e.g., a processor providing signal processing functionality) also may implement or support at least a portion of the monitoring functionality discussed herein. For example, a heart failure information acquisition component 1538 may perform information acquisition operations as described above with reference to FIGS. 3-7 and 10-12. In addition, a heart failure indication generation component 1539 may perform indication generation operations as described above with reference to FIGS. 3-7 and 10-12.

It should be appreciated that various modifications may be incorporated into the disclosed embodiments based on the teachings herein. For example, the structure and functionality taught herein may be incorporated into types of devices other than the specific types of devices described above. In addition, various techniques may be used to acquire LAP information, temperature information, congestion information, and perfusion information in different implementations. Also, various algorithms or techniques may be employed to provide a heart failure indication in different implementations. Furthermore, a heart failure indication may take various forms in different implementations. For example, in some cases the described techniques for providing a 2-D indication of congestion/perfusion may be employed to provide a 2-D indication of LAP/temperature. Conversely, the described techniques for providing state-based indication generation for LAP/temperature may be employed to provide state-based indication generation for congestion/perfusion.

The various structures and functions described herein may be incorporated into a variety of apparatuses and implemented in a variety of ways. Different embodiments of such an apparatus may include a variety of hardware and software processing components. In some embodiments, hardware components such as processors, controllers, state machines, logic, or some combination of these components, may be used to implement the described components or circuits.

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium).

Moreover, some of the operations described herein may be performed by a device that is located externally with respect to the body of the patient. For example, an implanted device may send raw data or processed data to an external device that then processes the received data.

The components and functions described herein may be connected or coupled in many different ways. The manner in which this is done may depend, in part, on whether and how the components are separated from the other components. In some embodiments some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, light pulses transmitted through an optical medium such as an optical fiber or air, or RF waves transmitted through a medium such as air, and so on. In addition, a plurality of signals may be collectively referred to as a signal herein. The signals discussed above also may take the form of data. For example, in some embodiments an application program may send a signal to another application program. Such a signal may be stored in a data memory.

Moreover, the recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise a set of elements may comprise one or more elements.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above it will be understood that the teachings herein are intended to cover any changes, adaptations or modifications which are within the scope of the disclosure.

What is claimed is:

1. An implantable medical apparatus, comprising:
a heart failure information acquisition circuit configured to collect temperature information and left atrial pressure information to provide data sets for an alarm state, a normal sleep state, and a normal wake state; and
a heart failure indication generator configured to:
determine whether a set of temperature and left atrial pressure information collected by the heart failure information acquisition circuit corresponds to the alarm state,
update a heart failure score based on the determination, and
generate at least one indication of heart failure status if the heart failure score meets at least one threshold.

2. The apparatus of claim 1, wherein:
the at least one threshold comprises a first threshold and a second threshold;
the at least one indication of heart failure status comprises a first type of indication and a second type of indication; and
the generation of the at least one indication comprises generating the first type of indication if the heart failure score meets the first threshold and generating the second type of indication if the heart failure score meets the second threshold.

3. The apparatus of claim 2, wherein:
the first threshold corresponds to an acute heart failure condition associated with higher than normal left atrial pressure and normal temperature; and
the second threshold corresponds to a chronic heart failure condition associated with higher than normal left atrial pressure and lower than normal temperature.

4. The apparatus of claim 2, wherein:
the first type of indication comprises an indication of possible onset of heart failure; and
the second type of indication comprises an indication of a need for immediate medical attention.

5. The apparatus of claim 2, wherein:
the first threshold comprises a first defined quantity indicative of how many recent occurrences of temperature and left atrial pressure information correspond to the alarm state;
the second threshold comprises a second defined quantity indicative of how many recent occurrences of temperature and left atrial pressure information correspond to the alarm state; and
the second defined quantity corresponds to a larger number of recent occurrences than the first defined quantity.

6. The apparatus of claim 1, wherein the updating of the heart failure score comprises:
increasing the heart failure score if the determination determined that there has been a transition from the alarm state to the wake or sleep state; and
decreasing the heart failure score if the determination determined that there has been a transition from the wake or sleep state to the alarm state.

7. The apparatus of claim 1, wherein the updating of the heart failure score comprises incrementing the heart failure score by a defined amount if temperature information collected by the heart failure information acquisition circuit indicates that patient temperature is below a temperature threshold during an alarm state.

8. The apparatus of claim 1, wherein the determination comprises comparing the set of temperature and left atrial pressure information with at least one of the group consisting of: the alarm state data set, the sleep state data set, and the wake state data set.

9. The apparatus of claim 1, further comprising a temperature sensor configured to provide the temperature information.

10. The apparatus of claim 1, further comprising a pressure sensor configured to provide the left atrial pressure information.

11. A cardiac monitoring method, comprising:
collecting temperature information and left atrial pressure information to provide data sets for an alarm state, a normal sleep state, and a normal wake state;
determining whether a set of the collected temperature and left atrial pressure information corresponds to the alarm state;
updating a heart failure score based on the determination; and
generating at least one indication of heart failure status if the heart failure score meets at least one threshold.

12. The method of claim 11, wherein:
the at least one threshold comprises a first threshold and a second threshold;
the at least one indication of heart failure status comprises a first type of indication and a second type of indication; and
the generation of the at least one indication comprises generating the first type of indication if the heart failure score meets the first threshold and generating the second type of indication if the heart failure score meets the second threshold.

13. The method of claim 12, wherein:
the first threshold corresponds to an acute heart failure condition associated with higher than normal left atrial pressure and normal temperature; and
the second threshold corresponds to a chronic heart failure condition associated with higher than normal left atrial pressure and lower than normal temperature.

14. The method of claim 12, wherein:
the first type of indication comprises an indication of possible onset of heart failure; and
the second type of indication comprises an indication of a need for immediate medical attention.

15. A cardiac monitoring method, comprising:
collecting congestion information;
collecting perfusion information; and
providing a two-dimensional indication of a heart failure trend based on the collected congestion and perfusion information.

16. The method of claim 15, wherein the two-dimensional indication comprises chronological heart failure status information.

17. The method of claim 16, wherein the providing of the two-dimensional indication comprises displaying a two-dimensional plot where the congestion information corresponds to a first axis of the two-dimensional plot and the perfusion information corresponds to a second axis of the two-dimensional plot.

18. The method of claim 15, wherein the collection of the congestion and perfusion information comprises acquiring multiple instances of congestion and perfusion information over a period of time.

19. The method of claim 15, wherein the collected congestion information is derived from evoked response information.

20. The method of claim 15, wherein the collected perfusion information is derived from arteriole stiffness information.
